(19) United States
(12) Patent Application Publication
(10) Pub. No.: US 2008/0114038 A1
(43) Pub. Date: May 15, 2008

(54) Drugs Derived from Diclofenac Containing No-Donor Heterocycles, Composition and Method of Inflammation Treatment

(76) Inventors: Jose Pedrazzoli, Campinas (BR); Paulo Sergio De Carvalho, Campinas (BR); Alessandra Gambero, Campinas (BR)

Correspondence Address: MEREK, BLACKMON & VOORHEES, LLC, 673 S. WASHINGTON ST., ALEXANDRIA, WV, 22314, US

(21) Appl. No.: 11/665,734

(22) PCT Filed: Sep. 23, 2005
(86) PCT No.: PCT/BR05/00202

§ 371 Date: Jun. 12, 2007

(30) Foreign Application Priority Data
Oct. 21, 2004 (BR) 0405520-9

(51) Int. Cl. A61K 31/41; A61P 1/00

(52) U.S. Cl. 514/364

(57) ABSTRACT

The present invention refers to drugs resulting from the pharmaceutical substance diclofenac, relative to the formula. The invention further refers to a pharmaceutical composition, which involves the said drugs and a pharmaceutically adequate vehicle. It is also described a method for the treatment of inflammation through the administration of the new drugs in patients with gastric problems or subjected to long-term treatments.

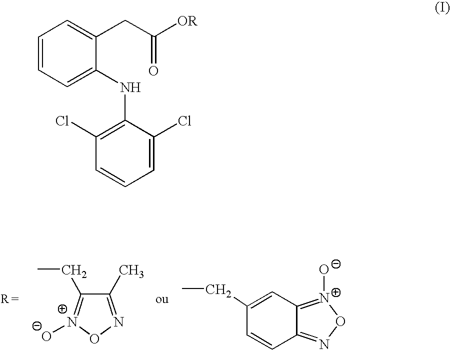

### FIELD OF THE INVENTION

The present invention refers to the accomplishment of new drugs with anti-inflammatory activities, for the application in patients with gastric problems or submitted to long-term periods. More specifically, the new drugs are substances derived from the diclofenac drug. Said drugs are non-steroidal anti-inflammatory substances that present, in their structures, a heterocyclic portion of the chemical class of furoxan or of benzofuroxan, derived from the incorporation of the respective heterocycle to the original structure of the diclofenac.

### STATE OF THE ART

In the past, a few people were known as healers due to the fact that they used balsams or infusions made of natural plants to treat people who presented pain, fever and swelling in any part of the body. These symptoms are characteristic clinical signs of an inflammatory process and certain plants have properties capable of relieving these symptoms. Thus, the healers instinctively created the first anti-inflammatory drugs in the history of mankind.

The inflammatory process is considered as the response of the organism to a tissue aggression. This process usually occurs accompanied by clinical signs, such as warmth, pain, edema, tumor and loss of function in the impaired organ.

The chronic inflammatory process can be treated by different therapeutic procedures, in view of its complexity and the diversity of physiological mediators involved.

The most classical form of treatment applied to the inflammatory processes includes the employment of non-steroidal anti-inflammatory agents (AINEs), which have the property of blocking a specific enzyme, called cycloxigenase (COX). The cycloxigenase can be divided into two groups, in accordance with the variation that it occurs in the same. Said groups of cycloxigenase are: COX-1 and COX-2.

The cycloxigenase, COX-1, is found all over our organism and is part of the cellular envelope. Its present is very important in several regions of our organisms, such as, for example, the stomach and the kidneys. COX-1, located in the stomach, produces a number of substances, such as, for example, the prostaglandins, which protect and prevent the emergence of ulcers, hemorrhages and stomach perforations. This cycloxigenase, COX-1, is also very important for the kidneys, since it produces substances that control the adequate receipt of blood in this organ, thus keeping its operation.

The cycloxigenase, COX-2, under normal physiological conditions, does not work; however, when any one organ in our organism suffers some kind of impairment, the cycloxigenase, COX-2, turns up and starts the production of substances, which will develop the inflammatory process and the pain.

The inhibition of cycloxigenases, COX-1 and COX-2, also known as prostaglandin-endoperoxide synthase (PGHS), results in the inhibition of the transformation of the arachidonic acid released by the phospholipase (PLA) in prostaglandins (PG's).

The AINEs include not only the class of the anti-inflammatory drugs, but also the class of the analgesics, of the anti-pyretic and the anti-thrombotic. The AINEs make up one of the largest factors of aggression for the gastrointestinal mucous membrane, just as they are some of the kinds of medicines most widely used in self-medication.

The prostaglandins are also responsible for the sensibilization of the local pain receptors and the central neurons of the spinal medulla.

One of the drugs known in the state of the art as having anti-inflammatory activity is the diclofenac. The diclofenac is an efficient anti-inflammatory agent in the treatment of the inflammatory processes; however, its use is recommended for not too long treatments in patients who do not present previous histories of gastrointestinal problems.

The diclofenac drug has a high degree of ulcerogenic action, since, as it inhibits the prostaglandins, it reduces the protections to the stomach mucous membranes due to the reduction of the blood flow with the subsequent reduction of the production of the protecting mucosa in the stomach.

That way, the preparations of new drugs that have similar anti-inflammatory efficiency as compared to that of the diclofenac drug and that have less aggressive effects on the stomach mucosa are the object of constant research.

Several modifications of the anti-inflammatory agents have been studied. One of these modifications involves the nitration of organic functions for the achievement, of derivative nitrates capable of releasing nitrogen monoxide (NO) under physiological conditions. This oxide has a vasodilating activity and is capable of promoting the protection of the gastric mucosa against the anti-inflammatory agents that cause the impairment of the stomach walls.

An example of this methodology is found in the North American U.S. Pat. No. 6,083,515. In patent US'515, it is described a non-steroidal anti-inflammatory drug substituted by nitrogen monoxide (NO) or a donating compound, releasing or transferring nitrogen monoxide (NO) and methods of inflammation treatment, pain, injuries, gastrointestinal injuries and/or fever so as to use the protecting composition against gastrointestinal injuries, renal injuries and other toxicities that were induced by non-steroidal anti-inflammatory drugs.

This way, the present invention aims at developing compounds that are analogous to the diclofenac, with anti-inflammatory activity and properties that are less aggressive to the stomach mucous membrane.

### SUMMARY OF THE INVENTION

The present invention refers to the development of new drugs with confirmed anti-inflammatory activity. More specifically, the new drugs are substances derived from the drug diclofenac, which presents a modification in a portion of its structure.

Said drugs are non-steroidal compounds that have, in their structural skeleton, heterocyclic substituents from the chemical classes of furoxan or benzofuroxan, as an ester function resulting from the chemical reaction of the diclofenac with furoxanyl-alkyl halides or furoxanyl-alkyl halides, as per formula below. This heterocyclic portion is capable of, under physiological conditions, releasing monoxide of nitrogen (NO) and thus, develop its vasodilating capacity and promote the protection of the stomach mucous membrane against gastrointestinal injuries.

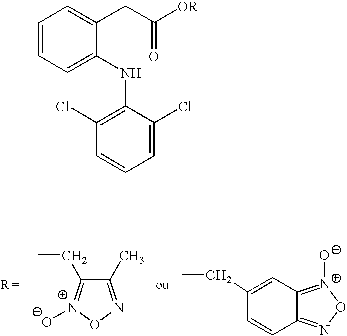

### BRIEF DESCRIPTION OF THE FIGURES

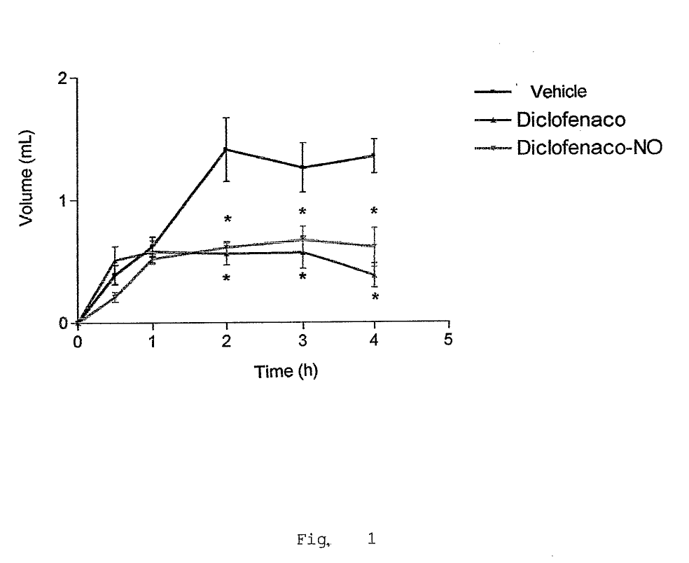

In FIG. 1, the curves to the volume of the paw after the intraplantary administration of carrageenan in animals previously treated with the vehicle (DMSO 10%), 10 mg/kg of diclofenac and equivalent molar dose of modified diclofenac.

### DETAILED DESCRIPTION OF THE INVENTION

For the development of new diclofenac drugs, the modified diclofenacs, first of all it is necessary to have the synthesis of two predecessor compounds, which will be the reagent substances of the syntheses of the new drugs, the modified diclofenacs.

The predecessor compounds to be synthesized are: a furoxanyl bromide and a benzofuroxanyl bromide.

### EXAMPLE 1

The compound 3,4-dimethyl-furazan-2-oxide was prepared through the addition of approximately 500 mg of dimethylglyoxime to a solution previously prepared by the dissolution of approximately 173 mg of sodium hydroxide in approximately 5 mL of ethanol 95%.

After that, the mixture was added by approximately 6 mL of a solution of sodium hypochlorite 10%. This solution of sodium hypochlorite was added drop by drop under constant magnetic stirring and ice bath.

After the addition of the sodium hypochlorite solution, the reaction mixture was kept under stirring during an approximate time period of 40 minutes. During this period of time, the reaction mixture was under constant stirring and said mixture remained under refrigeration by means of ice bath.

After stirring, the mixture was extracted with approximately 15 mL of dichloromethane by means of a separatory funnel. The extraction procedure is repeated preferably three consecutive times.

The organic phases derived from the stages of extraction were mixed and the resulting solution was washed once with a solution saturated with sodium chloride and dried by the addition of approximately 2 g of anhydrous sodium sulfate.

The anhydric sodium sulfate was removed from the mixture by means of adequate techniques of mixture separation. In the present case, the technique used for the separation of the anhydric sodium sulfate was filtration and the technique used for the separation of the dichloromethane was evaporation.

The product 3,4-dimethyl-furazan-2-oxide was obtained as a clear oil. The output of the synthesis reaction of the 3,4-dimethyl-furazan-2-oxide was approximately 66%.

The purity of the compound 3,4-dimethyl-furazan-2-oxide was checked by means of analysis of appropriate techniques. In the present case, the analysis technique was the gaseous chromatography coupled with mass spectrometry (GCMS). The data of mass spectrometry obtained for the synthesized compound 3,4-dimethyl-furazan-2-oxide were: m/z (%) 114 (58), 84 (100), 66 (8), 54 (47), 53 (63), 51 (22), 42 (20).

### EXAMPLE 2

### Synthesis of the 3-bromomethyl-4-methyl-furazan-2-oxide, the Furoxanyl Bromide

The compound obtained in Example 1, 3,4-dimethyl-furazan-2-oxide was converted into 3-bromomethyl-4-methyl-furazan-2-oxide, the furoxanyl bromide. The reaction of conversion was performed by means of the addition of approximately 487 mg of N-bromosuccinimide and approximately 55 mg of benzoyl peroxide to a previous prepared solution, in which approximately 260 mg of 3,4-dimethyl-furazan-2-oxide was dissolved in approximately 10 mL of carbon tetrachloride.

The reaction mean was refluxed under magnetic stirring during a preferred period of eight hours, being, after this period of time, cooled preferably to a temperature between 20 to 25° C.

The solid that was formed, the succinimide, was removed by means of adequate mixture separatory techniques. In the present case, the technique used was filtration.

After the separation procedure, the solid residue was washed with approximately 3 mL of carbon tetrachloride solution and the organic solutions were mixed. The carbon tetrachloride was separated from the mixture by means of the evaporation technique so that the gross product, 3-bromethyl-4-methyl-furazan-2-oxide, could be obtained.

The product 3-bromomethyl-4-methyl-furazan-2-oxide was purified by the use of adequate techniques. The technique used was preferably the flash chromatography, in which it is used ethyl acetate/hexane 10% as aluent mixture.

The product 3-bromomethyl-4-methyl-furazan-2-oxide was obtained as a light yellow solid. The output of the synthesis reaction of the 3-bromomethyl-4-methyl-furazan-2-oxide was approximately 78%.

The purity of the compound was verified by means of the analysis of adequate techniques. In the present case, the analysis technique was the gaseous chromatography coupled with mass spectrometry (GCMS). The data of mass spectrometry obtained for the synthesized compound 3-bromomethyl-4-methyl-furazan-2-oxide were: m/z (%) 194 (9), 192 (9), 164 (12), 162 (12), 134 (5), 132 (5), 113 (24), 53 (100).

As a result of the complexity in the nomenclature of the synthesized compound 3-bromomethyl-4-methyl-furazan-2-oxide; from now on we will refer to it as furoxanyl bromide.

### EXAMPLE 3

The compound 5-methyl-benzo[1,2,5]oxadiazole-1-oxide was prepared by the dissolution of approximately 1.52 g of 4-methyl-2-nitro-aniline in a previously prepared solution by means of the dissolution of approximately 440 mg of sodium hydroxide in approximately 15 mL of ethanol 95%.

The mixture was cooled in ice bath and approximately 20 mL of a solution of sodium hypochloride 10% was added drop by drop to the mixture under constant magnetic stirring.

After the addition of the sodium hypochloride 10% solution, the reaction mean remained under constant stirring for a time period of approximately 40 minutes under refrigeration in water bath.

After this period of time, the precipitate that was formed was separated by means of adequate mixture separation techniques. In the present embodiment, the technique used was the filtration by suction, being afterwards washed with approximately 10 mL of cold water, which was subsequently removed from the solid residue by means of the suction technique.

The purification of the product was performed by means of the dissolution in approximately 50 mL of ethanol solution 70%, at a preferred temperature of 70° C.

The reaction mixture was left to rest under constant monitoring of the temperature by means of an adequate instrument, such as, for instance, a thermometer, until the temperature of the mixture dropped to approximately 25° C.

The synthesized product, 5-methyl-benzo[1,2,5]oxadiazole-1-oxide, was obtained in the form of yellow micro-needles. The output of the synthesis reaction of the 5-methyl-benzo[1,2,5]oxadiazole-1-oxide was of approximately 79%.

The purity of the compound was verified by means of an adequate technique of analysis. In the present case, the technique of analysis was the gaseous chromatography coupled with mass spectrometry (GCMS). The data of mass spectrometry obtained for the synthesized compound 5-methyl-benzo[1,2,5]oxadiazole-1-oxide were: m/z (%) 150 (87), 134 (38), 105 (27), 89 (69), 77 (59), 65 (100), 63 (40), 51 (42).

### EXAMPLE 4

### Synthesis of the 5-bromomethyl-benzo[1,2,5]oxadiazole-1-oxide, the Benzofuroxanyl Bromide.

The compound obtained in Example 3, 5-methyl-benzo[1,2,5]oxadiazole-1-oxide was converted into 5-bromomethyl-benzo[1,2,5]oxadiazole-1-oxide.

The conversion reaction was performed by means of the addition of approximately 1.34 g of N-bromosuccinimide and approximately 151 mg of benzoyl peroxide at a previously prepared solution by means of the dissolution of approximately 939 mg of 5-methyl-benzo[1,2,5]oxadiazole-1-oxide in approximately 60 mL of carbon tetrachloride.

The reaction mean was refluxed under constant magnetic stirring during an approximate period of time of five hours. After stirring, the mixture was cooled at a preferred temperature in the range of 20 to 25° C.

The solid formed, the succinimide, was removed from the reaction mean through adequate techniques of mixture separation. In the present embodiment, the technique used was filtration.

After the procedure of mixture separation, the solid residue was washed with approximately 10 mL of carbon tetrachloride and the organic solutions was mixed. The carbon tetrachloride was eliminated from the solid residue by means of the evaporation technique.

The crude product, 5-bromomethyl-benzo[1,2,5]oxadiazole-1-oxide, was purified by the use of adequate techniques. The technique used was preferably the flash chromatography, in which acetate of ethyl/hexane 5% is used as aluent mixture.

The product 5-bromomethyl-benzo[1,2,5]oxadiazole-1-oxide was obtained as a yellow solid. The output of the synthesis reaction of the 5-bromomethyl-benzo[1,2,5]oxadiazole-1-oxide was of approximately 85%.

The purity of the compound was verified by means of the analysis of adequate techniques. In the present case, the analysis technique was the gaseous chromatography coupled with mass spectrometry ((GCMS). The mass spectrometry data obtained for the synthesized compound 5-bromomethyl-benzo[1,2,5]oxadiazole-1-oxide were: m/z (%) 230 (12), 228 (12), 149 (100), 133 (60), 103 (13), 89 (76), 76 (30), 63 (66).

As a result of the complexity in the nomenclature of the synthesized compound 5-bromomethyl-benzo[1,2,5]oxadiazole-1-oxide, from now on we will refer to it as benzofuroxanyl bromide.

### General Procedure for the Synthesis of the Modified Diclofenacs

The modified diclofenacs, which contain at least a substituent of the chemical class of the furoxan or benzofuroxan, were synthesized by means of the reaction between the diclofenac and the furoxanyl bromide or between the diclofenac and the benzofuroxanyl bromide. The compounds furoxanyl bromide and benzofuroxanyl bromide were previously synthesized

### EXAMPLE 5

### Synthesis of the Modified Diclofenac by Means of the Furoxanyl Bromide

For a solution that was previously prepared by means of the dissolution of approximately 250 mg of sodium diclofenac in approximately 7 mL of anhydrous dimethylformamide (DMF), approximately 152 mg of furoxanyl bromide were added.

The resulting solution was stirred by means of an adequate equipment, such as, for example, a magnetic stirrer at a preferred temperature between 20 and 25° C. during a period of time of approximately two hours. After this period of time, 20 mL of distilled water were added and the mixture remained under constant stirring during approximately five more minutes.

After the stirring stage, the process of mixture extraction was performed with approximately 15 mL of a solution of dichloromethane by means of a separatory funnel. The extraction process was repeated preferably three times.

The organic phases derived from the extractions were mixed and the resulting solution was washed once with a saturated solution of sodium chloride and subsequently dried by the addition of approximately 2 g of anhydrous sodium sulfate.

The anhydrous sodium sulfate was removed from the mixture by means of adequate techniques of mixture separation. In the present embodiment, the technique used for the separation of the anhydrous sodium sulfate was the filtration and for the separation of the dichloromethane, it was used the evaporation to obtain the crude product with traces of DMF.

The crude product was purified through the use of adequate techniques. The technique used was preferably the flash chromatography, in which ethyl acetate and hexane 10-20% are used as aluent mixture.

The product [2-(2,6-dichloro-phenylamino)-phenyl]-acetic acid 4-methyl-2-oxy-furazan-3-ylmethyl ester was obtained as a white solid. The output of the reaction was of approximately 93%.

The purity of [2-(2,6-dichloro-phenylamino)-phenyl]-acetic acid 4-methyl-2-oxy-furazan-3-ylmethyl ester was verified by means of techniques of adequate analyses. In the present case, the analysis technique was the gaseous chromatography coupled with mass spectrometry (GCMS). The mass spectrometry data obtained [2-(2,6-dichloro-phenylamino)-phenyl]-acetic acid 4-methyl-2-oxy-furazan-3-ylmethyl ester were: m/z (%)409 (17), 407 (26), 352 (6), 350 (9), 277 (15), 242 (38), 214 (100), 179 (15), 151 (10), 89 (13), 77 (8).

### EXAMPLE 6

### Synthesis of the Modified Diclofenac by Means of the Benzofuroxanyl Bromide

For a solution that was previously prepared by means of the dissolution of approximately 250 mg of sodium diclofenac in approximately 7 mL of anhydrous dimethylformamide (DMF) approximately 180 mg of benzofuroxanyl bromide were added.

The resulting solution was stirred by means of an adequate equipment, such as, for example, a magnetic stirrer at a preferred temperature between 20 and 25° C. during a period of time of approximately two hours. After this period of time, 20 mL of distilled water were added and the mixture remained under constant stirring during approximately five more minutes.

After the stirring stage, the process of extraction of the mixture was performed with approximately 15 mL of a solution of dichloromethane by means of a separatory funnel. The extraction process was repeated preferably three times.

The organic phases derived from the extractions were mixed and the resulting solution was washed once with a saturated solution of sodium chloride and subsequently dried by the addition of approximately 2 g of anhydrous sodium sulfate.

The anhydrous sodium sulfate was removed from the mixture by means of adequate techniques of mixture separation. In the present embodiment, the technique used for the separation of the anhydrous sodium sulfate was the filtration and for the separation of the dichloromethane, the evaporation was used to obtain the crude product with traces of DMF.

The crude product was purified by the use of adequate techniques. The technique used was preferably the flash chromatography, in which ethyl acetate and hexane 10-20% are used as aluent mixture.

The product [2-(2,6-dichloro-phenylamino)-phenyl]-acetic acid 1-oxy-benzo[1,2,5]oxadiazol-5-ylmethyl ester was obtained as a light yellow solid. The output of the reaction was of approximately 89%.

The purity of the [2-(2,6-dichloro-phenylamino)-phenyl]-acetic acid 1-oxy-benzo[1,2,5]oxadiazol-5-ylmethyl ester was verified by means of the analysis of adequate techniques. In the present case, the analysis technique was the gaseous chromatography coupled with mass spectrometry (GCMS). The mass spectrometry data obtained for [2-(2,6-dichloro-phenylamino)-phenyl]-acetic acid 1-oxy-benzo[1,2,5]oxadiazol-5-ylmethyl ester were; m/z (%) 444 (1), 429 (13), 427 (18), 277 (11), 242 (26), 214 (100), 179 (15), 151 (10), 133 (9), 90 (9), 76 (15).

Although the compounds were synthesized by the synthetic routes herein described, the synthesis of these compounds is not limited to them, so that a specialist in the matter may decide to synthesize the same compounds by means of different synthetic routes, which were not mentioned in the present report.

The synthesized diclofenac derivatives, [2-(2,6-dichloro-phenylamino)-phenyl]-acetic acid 4-methyl-2-oxy-furazan-3-ylmethyl ester and [2-(2,6-dichloro-phenylamino)-phenyl]-acetic acid 1-oxy-benzo[1,2,5]oxadiazol-5-ylmethyl ester, were tested as to their pharmacological activities.

Below, we will describe the procedures of the pharmacological activity tests in vitro and in vivo and the results obtained for the referred to tests.

### Analysis of the Pharmacological Activity

### EXAMPLE A

### “Whole Blood Assay”—WBA—Method

This assay has been extensively used for the evaluation of potencies and activities of AINEs, so as to present the advantage as to its use when used in human cells rapidly obtained and so as to take into consideration the connection of the drug to plasmatic proteins.

The WBA assay consists of the previous dissolution of a sample of the diclofenac drug into a solution of DMSO 0.1% (v/v), so as to reach a final concentration in the sample of the diclofenac drug of approximately 0.01, 1 and 100 μM, and in the dissolution of a sample of the modified drug. In the present case, [2-(2,6-dichloro-phenylamino)-phenyl]-acetic acid 1-oxy-benzo[1,2,5]oxadiazol-5-ylmethyl ester, in a solution of DMSO 10% (v/v), so as to reach a final concentration in the sample of the modified drug of approximately 0.01, 1 and 100 μM.

Each one of the mixtures developed was stored in glass containers appropriate for centrifuging, such as, for example, assay tubes. In each one of the tubes, the solution formed by the mixtures totaled a final volume of approximately 10 μL.

At this stage, control samples were also stored with vehicle DMSO 10% (v/v). The control samples consist of an amount of the diclofenac drug added to a solution of DMSO 0.1% (v/v) and at a certain amount of the drug, modified diclofenac. In the present case, [2-(2,6-dichloro-phenylamino)-phenyl]-acetic acid 1-oxy-benzo[1,2,5]oxadiazol-5-ylmethyl ester to a solution of DMSO 0.1% (v/v).

### WBA Assay for COX-1:

A sample of approximately 20 mL of blood was collected from volunteers evaluated as being healthy conditions. The characterization of the healthy conditions took place by the fact that the people had not received any kind of medication during a preferred period of 14 days.

The sample of blood was collected without the use of an anti-coagulant* substance, by means of a technique of venous puncture and by means of adequate equipment, such as, for example, a plastic syringe and a disposable needle.

Aliquots of approximately 1 mL of the blood sample collected were immediately transferred from the plastic syringe to appropriate containers, such as, for example, glass assay tubes, appropriate for centrifuging, which had different concentrations of the modified diclofenacs or of the vehicle, solution of DMSO 10%, all in a volume of 10 μl. The final concentration of the modified diclofenacs in each assay tube was of approximately 0.01; 1 and 100 μM respectively.

After the stage of adding the blood aliquot to the assay tubes, the referred assay tubes were incubated at a preferred temperature of 37° C., in adequate equipment, such as, for example, a hothouse, for a preferred period of one hour.

Following the incubation stage, the assay tubes were stirred, preferably by the centrifugation technique, for a period of approximately ten minutes at a preferred rotation of approximately 2,000 rpm.

After the centrifugation of the assay tubes, the serum of the blood samples was collected with the help of an adequate instrument. In the present embodiment, a Pasteur pipette was used. The solid residue was adequately disposed of.

The serum collected was frozen at a preferred temperature of −20° C. until the dosage of tromboxane B2 (TXB2) was used. The dosage of the tromboxane B2 is performed with the use of the enzymatic immunoassay commercial kit.

### WBA Assay for COX-2:

A sample of blood of approximately 20 mL was collected from volunteers evaluated as being in perfectly healthy conditions. The characterization of the healthy conditions took place by the fact that the people having received no kind of medication during a preferred period of fourteen days.

Contrary to the WBA assay for COX-1, in this assay, the blood sample was collected with the use of an anticoagulant substance, preferably heparin (10 U.I/mL), by means of a venous puncture technique and adequate equipment, such as, for example, a plastic syringe and a disposable needle.

Aliquots of approximately 1 mL of the blood sample collected were immediately transferred from the plastic syringe to appropriate containers. In the present embodiment, assay tubes of polypropylene were used, which contained the modified diclofenacs at a final concentration of approximately 0.01, 1 and 100 μM or of the vehicle, a solution of DMSO at 10% all at a final volume of approximately 10 μl. After that, approximately 10 μL of a solution of lipopolyssacaride (LPS) were added to the polypropylene assay tubes, at the preferred concentration of 10 μg/mL. The LPS solution is a powerful inducer of COX-2 in leukocytes. The tubes were incubated at a preferred temperature of 37° C., in adequate equipment, such as, for example, a hothouse, for a preferred period of 24 hours.

After the incubation stage, the assay tubes were stirred preferably by the centrifugation technique, for a period of approximately ten minutes at a preferred rotation of approximately 2,000 rpm.

After the stage of centrifugation of the polypropylene assay tubes, the plasma of the blood samples was collected with the help of an adequate instrument. In the present embodiment, a Pasteur pipette was used. The solid residue was adequately disposed of.

The plasma collected was frozen at a preferred temperature of approximately −20° C. until the dosage of prostaglandin E₂ (PGE₂) was achieved. The dosage of the prostaglandin E₂ (PGE₂) was achieved by means of the use of the enzymatic immunoassay commercial kit.

### EXAMPLE B

### Edema of Rat Paws Induced by Carragenine: Characterization of the Anti-Inflammatory Activity In Vivo

For the performance of this one test, animals, such as, for example, male rats of the Wistar type were used, which weighed between 150-180 g. These rats were acquired from CEMIB/UNICAMP.

The animals were previously treated by intraperitoneal means (i.p.) with approximately 100 mg/kg of diclofenac, equivalent molar dose of the modified diclofenac ([2-(2,6-dichloro-phenylamino)-phenyl]-acetic acid 1-oxy-benzo[1,2,5]oxadiazol-5-ylmethyl ester) or vehicle (DMSO 10%). Five rats were treated with the diclofenac, five rats with the modified diclofenac and five rats with the DMSO vehicle.

After a period of approximately 30 minutes, the animals were inoculated with an intraplantary injection of approximately 0.1 mL of a solution of carragenine 1% in saline solution, at the right hind paw, while the contralateral paw received the same volume but only of the saline solution.

The quantification of the volume of the paw was performed by means of an adequate instrument, such as, for example a pletismometer. The volume of the paws was checked before the administration of the carragenine (T₀) and after 30 minutes, 1, 2, 3 and 4 hours. The T₀ value was subtracted from the other values arrived at.

### EXAMPLE C

### Analysis of the Relative Gastric Tolerance

For the test of gastric tolerance, animals were used such as, for example, male rates of the Wistar type, which weighed between 150-180 g; these animals were acquired from CEMIB/UNICAMP.

The animals were previously treated orally (gavage) with approximately 50 mg/kg of diclofenac, molar dose equivalent of the modified diclofenac ([2-(2,6-dichloro-phenylamino)-phenyl]-acetic acid 1-oxy-benzo[1,2,5]oxadiazol-5-ylmethyl ester) or vehicle (DMSO 10%). Five rats were treated with the diclofenac, five rats with the modified diclofenac and five rats with the DMSO vehicle.

After a period of approximately four hours, the animals were sacrificed and had their stomachs removed and opened at their largest curvature. By means of a microscopic evaluation, the Ulcerative Lesion Index (ULI) was established in accordance with the criteria below:

| | | |
|---|---|---|
| Loss of normal morphology | 1 | point |
| Discoloration of the mucous | 1 | point |
| Mucosa .edema | 1 | point |
| Hemorrhages | 1 | point |
| Spots of petechia (up to 9 spots) | 2 | points |
| Spots of petechia (>10) | 3 | points |
| Ulcers up to 1 mm | *n × 2 | points |
| Ulcers above 1 mm | *n × 3 | points |
| Perforated ulcers | *n × 4 | points |

OBS: *number of ulcers found.

### RESULTS

The process of blood coagulation is dependent on the production of plaquetary TXA2, product of the COX-1. Thus, the capacity of the diclofenac and the modified diclofenac to inhibit the activity of the COX-1 enzyme was verified by means of the seric determination of TXA2 after the spontaneous coagulation of aliquots of the blood sample previously exposed to different concentrations of the diclofenac and to different concentrations of the modified diclofenac.

With the results obtained, curves of inhibition in the production of TXA2 were built. Based on the referred curves, it was possible to calculate the concentration responsible for the inhibition of 50% of the production of TXA2 (IC50).

It can be noted that, after the incubation of an aliquot of the blood sample with the vehicle (DMSO), there followed the production of approximately 3630 ng/mL of TXA2. The previous exposure of an aliquot of the blood sample at approximately 40.41 μM of the modified diclofenac substance, leads to the inhibition of approximately 50% of the production of TXA2.

The same inhibition of approximately 50% is reached with the concentration of 1.37 M of standard diclofenac. These data show that the modified diclofenac has a lower capacity of inhibiting COX-1, when compared with the standard diclofenac. This fact suggests a higher selectivity of the isoforms of COX.

For the analysis of the anti-inflammatory activity of the modified diclofenac, curves of inhibition in the production of PGE2 were built after the dosage of the prostaglandin E₂ (PGE₂), with the results obtained. Based on the referred to curves, it was possible to calculate the concentration responsible for the inhibition of approximately 50% of the production of PGE2 (IC50).

From the results obtained, one comes to the conclusion that the value of IC50 for the production of PGE2 occurs with the concentration of approximately 6.58 μM of modified diclofenac.

With the standard drug, the diclofenac at the concentration of 6.51 μM, the same inhibiting effect of approximately 50% was obtained.

The results obtained show that the modified diclofenac retains its inhibiting capacity over the isoforms of COX.

The characterization of the in vivo anti-inflammatory activity was made by a classic model of inflammation, by means of the use of an experimental model of edema of paw induced by carragenine in rats. In FIG. 1, one can note that the modified diclofenac is capable of inhibiting the edema of the paw induced by carragenine in the same way that the standard diclofenac.

For the test of Gastric Tolerance, it was noted that the animals, after the acute oral administration of AINEs, presented the formation of ulcerative lesions in the gastric mucosa.

The oral administration of the drug, diclofenac, at the preferred doses between 10 and 50 mg/kg, let to ulceration in the gastric mucosa with an index, ULI, of approximately 8.17+/−1.22 and 16.50+/−1.85, respectively.

On the other hand, the administration of approximately 10 and 50 mg/kg of the drug, modified diclofenac, led to ulceration in the gastric mucosa with an ULI index of approximately 4.8±0.97 and 6.00±1.45, respectively.

The Gastric Ulceration Lesion Index (ULI) for the control group, which was not treated with the anti-inflammatory substances, but only with the solution of DMSO was of approximately 2.30±0.20.

With the analysis of the results disclosed, we come to the conclusion that the compounds of the present invention present a lower ulcerogenic activity (p<0.01) when compared to the standard compound of the state of the art.

Now, the present invention will be thoroughly described by means of examples. It must be pointed out that the invention is not restricted to these examples, but that it also includes variations and modifications within the limits in which it works.

The new drugs of the present invention are used in pharmaceutical compositions, which can be administered orally, as a powder, as tablets, as capsules, as pills, or in the form of emulsions, solutions or suspensions. The inactive components in this case include excipients, ligands, desintegrators, diluents, lubricants, etc.

The solid compositions contain the active ingredient in a mixture of non-toxic excipients appropriate for the manufacture of pills, such as starch, lactose, certain types of carbonates and bicarbonates, phosphates, talcum, etc. The pills may or not be coated.

In the case of suspensions, syrups or aqueous solutions, excipients such as methyl cellulose, sodium alginate, acacia starch, lecithin, etc. can be used and one or more additives, such as preserving agents, dyes, flavoring agents, thickeners, polyols, saccarose, glucose, etc.

The invention that is herein described, as well as the aspects covered must be considered as one of the possible embodiments. However, it must stand clear that the invention is not limited to these embodiments and those with skills in the technique will perceive that any particular characteristic introduced therein should only be understood as something that was described in order to facilitate the understanding and cannot be performed at the risk of driving away from the inventive concept described. The limiting characteristics of the object of the present invention are related to the claims that are part of the present description.

1. New drugs resulting from the pharmaceutical substance diclofenac, characterized by the formula:

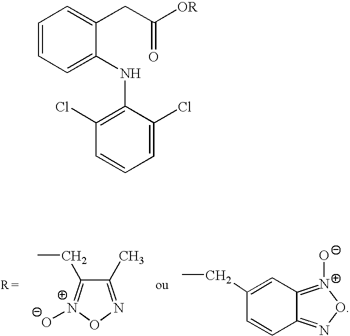

2. New drugs resulting from the pharmaceutical drug diclofenac, in accordance with claim 1, characterized by involving non-steroidal compounds, having in their structural skeleton, heterocyclical substituents.

3. New drugs resulting from the pharmaceutical drug diclofenac, in accordance with claim 1, characterized by the fact that their substituents are from the same chemical classes, furoxan or benzofuroxan, with an ester function resulting from the chemical reaction of the diclofenac with furoxanyl-alkyl halides or benzofuroxanyl-alkyl halides.

4. Pharmaceutical composition characterized by the fact that it involves the new drugs from claim 1 and a pharmaceutically adequate vehicle.

5. Pharmaceutical composition characterized by the fact that it involves the new pharmaceutical drugs defined in claim 1 and compounds that, under physiological conditions, release nitrogen monoxide (NO).

6. Pharmaceutical composition in accordance with claim 5, characterized by the fact that nitrogen monoxide promotes its vasodilating capacity and leads to the protection of the stomach mucosa from gastrointestinal lesions.

7. Method for the treatment of inflammation characterized by the administration of the new drugs defined in claim 1 in patients with duodenal gastric problems or subjected to long-term treatments.

8. Method of inflammation treatment according to claim 7, characterized by the amount of modified diclofenac administered preferably between approximately 1 and 50 mg/kg.

9. New drugs resulting from the pharmaceutical drug diclofenac, in accordance with claim 2, characterized by the fact that their substituents are from the same chemical classes, furoxan or benzofuroxan, with an ester function resulting from the chemical reaction of the diclofenac with furoxanyl-alkyl halides or benzofuroxanyl-alkyl halides.
